(19) United States
(12) Patent Application Publication
(10) Pub. No.: US 2025/0120593 A1
(43) Pub. Date: Apr. 17, 2025

(54) BIOMETRIC INFORMATION MEASUREMENT DEVICE

(71) Applicant: OMRON HEALTHCARE Co., Ltd., Kyoto (JP)

(72) Inventors: Kentaro MORI, Kyoto (JP); Takeshi KUBO, Kyoto (JP); Takashi ONO, Kyoto (JP); Kosuke ABE, Kyoto (JP); Shohei IWATA, Kyoto (JP)

(21) Appl. No.: 18/990,341

(22) Filed: Dec. 20, 2024

Related U.S. Application Data
(63) Continuation of application No. PCT/JP2023/004821, filed on Feb. 13, 2023.

(30) Foreign Application Priority Data
Jul. 22, 2022 (JP) 2022-117329

(51) Int. Cl. A61B 5/0205; A61B 5/00; A61B 5/021; A61B 5/28

(52) U.S. Cl. CPC A61B 5/0205; A61B 5/021; A61B 5/28; A61B 5/7475; A61B 5/681; A61B 2560/0468

(57) ABSTRACT

A technique for obtaining an electrocardiogram of good waveform quality. A biological information measurement device for measuring a blood pressure and an electrocardiographic waveform of a subject includes: a blood pressure measurement control unit configured to control measurement of a blood pressure of a measurement site of the subject; a first electrode to be in contact with a first site of the subject; a second electrode to be in contact with a second portion of the subject different from the first site; an electrocardiographic measurement control unit configured to control measurement of an electrocardiographic waveform of the subject through the first electrode and the second electrode; a main body portion including the blood pressure measurement control unit and the electrocardiographic measurement control unit; an instruction input portion operated by the subject to input an instruction; and a fixing portion configured to fix the main body portion to the measurement site.

### CROSS-REFERENCE TO RELATED APPLICATIONS

This application is the U.S. national stage application filed pursuant to 35 U.S.C. 365 (c) and 120 as a continuation of International Patent Application No. PCT/JP2023/004821, filed Feb. 13, 2023, which application claims priority to Japanese Patent Application No. 2022-117329, filed Jul. 22, 2022, which applications are incorporated herein by reference in their entireties.

### TECHNICAL FIELD

The present invention relates to a biological information measurement device.

### BACKGROUND ART

It has recently become common for an individual to measure information (hereinafter, also referred to as biological information) related to a body and health of the individual such as a blood pressure value and an electrocardiographic waveform on a daily basis by himself/herself by using a measurement device and utilize the measurement result for health management. For the viewpoint, a demand of portability-focused devices has increased, many portable measurement devices have been proposed, and portable devices that can measure biological information including an electrocardiographic waveform have also been proposed (see Patent Document 1).

Patent Document 1 discloses a technique of providing, in a wristwatch-type wearable electrocardiograph, an electrode around a front surface of a casing and increasing a degree of freedom of a contact position.

Patent Document 2 discloses a technique of providing, in a wristwatch-type wearable electrocardiograph, an electrode in a switch and clarifying a contact point.

### CITATION LIST

### Patent Literature

Patent Document 1: US 2019/0072912 A

### SUMMARY OF INVENTION

### Technical Problem

Unfortunately, using only the switch as the electrode as in Patent Document 2 limits a user's posture for measurement. In particular, a device for measuring a blood pressure simultaneously with an electrocardiogram needs the device to be adjusted to a height of a heart. Limiting a position of an electrode may cause an unreasonable posture in touching due to the body shape of the user or the like, may cause an unstable contact surface between a body of the user and the electrode, or may degrade the waveform quality of the electrocardiogram due to the influence of myoelectricity.

In view of the foregoing, it is an object of the present invention to provide a technique for obtaining an electrocardiogram of good waveform quality.

To solve the above problems, the present invention is a biological information measurement device for measuring a blood pressure and an electrocardiographic waveform of a subject and includes:
a blood pressure measurement control unit configured to control measurement of a blood pressure of a measurement site of the subject;
a first electrode to be in contact with a first site of the subject;
a second electrode to be in contact with a second site of the subject different from the first site;
an electrocardiographic measurement control unit configured to control measurement of an electrocardiographic waveform of the subject through the first electrode and the second electrode;
a main body portion including the blood pressure measurement control unit and the electrocardiographic measurement control unit;
an instruction input portion operated by the subject to input an instruction; and
a fixing portion configured to fix the main body portion to the measurement site
The main body portion includes a casing including a side wall portion surrounding an entire circumference of the main body portion from an outer circumference side when a direction facing the measurement site is defined as an axial direction.
The first electrode includes the side wall portion and the instruction input portion.

Thus, when the subject attempts to operate, to measure the electrocardiographic waveform, an operation button by means of the first site while causing the first site to contact the side wall portion constituting the first electrode, because the first electrode is configured to include the operation button, a change in a contact state between the subject and the first electrode due to the operation of the operation button can be suppressed, the contact state can be stabilized, and an electrocardiogram of good waveform quality can be obtained. The side wall portion can be formed in appropriate shapes such as a square and a circle and is not limited to these shapes.

In the present invention, the instruction input portion may be provided independently of the side wall portion.

Thus, even when the user operates the instruction input portion provided independently of the side wall portion in attempting to contact the side wall portion in order to measure the electrocardiogram, a change in a contact state between the first site of the user and the first electrode is suppressed, and the contact state is stabilized.

In the present invention, the instruction input portion may include the side wall portion.

Thus, when attempting to contact the side wall portion in order to measure the electrocardiogram, the user can input the instruction without changing the contact state with the side wall portion, thus stabilizing the contact state between the first site of the user and the first electrode.

In the present invention, the side wall portion may include a concave side wall portion which is concave toward an inner circumference side when the direction facing the measurement site is defined as the axial direction.

Thus, the first site of the user is less likely to slip with respect to the concave side wall portion due to the shape of the concave side wall portion, thus a change in the contact state between the first site of the user and the first electrode including the concave side wall portion is suppressed, and the contact state is stabilized.

In the present invention, an uneven portion may be formed on a surface of the side wall portion.

Thus, the first site of the user is less likely to slip with respect to the side wall portion due to the uneven portion of the side wall portion, thus a change in the contact state between the first site of the user and the first electrode including the side wall portion is suppressed, and the contact state is stabilized.

In the present invention, the instruction may be an instruction for the measurement of the blood pressure.

Thus, when the subject attempts to operate, to measure the electrocardiographic waveform, an operation button for inputting an instruction for blood pressure measurement by means of the first site while causing the first site to contact the side wall portion constituting the first electrode, because the first electrode is configured to include the operation button, in measuring in parallel the blood pressure and the electrocardiographic waveform, a change in the contact state between the subject and the first electrode due to the operation of the operation button can be suppressed, and the contact state can be stabilized.

According to the present invention, it is possible to provide a technique for obtaining an electrocardiogram of good waveform quality.

### BRIEF DESCRIPTION OF DRAWINGS

FIG. 1 is a diagram illustrating an appearance of a biological information measurement device according to Example 1.

FIG. 2 is a diagram illustrating the appearance of the biological information measurement device according to Example 1 when the device is mounted.

FIG. 3 is a functional block diagram of the biological information measurement device according to Example 1.

FIG. 4 is a cross-sectional view of a cuff assembly portion of the biological information measurement device according to Example 1.

FIG. 5(A) is a diagram illustrating the configuration of each part when the biological information measurement device according to Example 1 is mounted, and FIG. 5(B) is a diagram illustrating the arrangement of electrodes in particular.

FIG. 6 is a diagram illustrating an electrical connection relationship in the biological information measurement device according to Example 1.

FIG. 7 is a diagram illustrating an electrical connection of a casing of the biological information measurement device according to Example 1.

FIG. 8 is a diagram illustrating an electrical connection of a switch of the biological information measurement device according to Example 1.

FIG. 9 is a diagram describing the effect of the biological information measurement device according to Example 1 .

FIG. 10 is a diagram illustrating a measurement posture using the biological information measurement device according to Example 1.

FIG. 11 is a diagram illustrating an electrical connection of a switch of a biological information measurement device according to a modified example of Example 1.

FIGS. 12(A) to 12(C) are diagrams illustrating a configuration of a casing of a biological information measurement device according to Example 2.

FIGS. 13(A) to 13(C) are diagrams illustrating a configuration of a biological information measurement device according to Example 3.

### DESCRIPTION OF EMBODIMENTS

Embodiments of the present invention will be specifically described below with reference to the drawings.

### Example 1

Hereinafter, an example of the embodiments of the present invention will be described. It should be noted that the dimension, material, shape, relative arrangement, and the like of the components described in the present examples are not intended to limit the scope of this invention to them alone, unless otherwise stated.

### Overall Configuration of Device

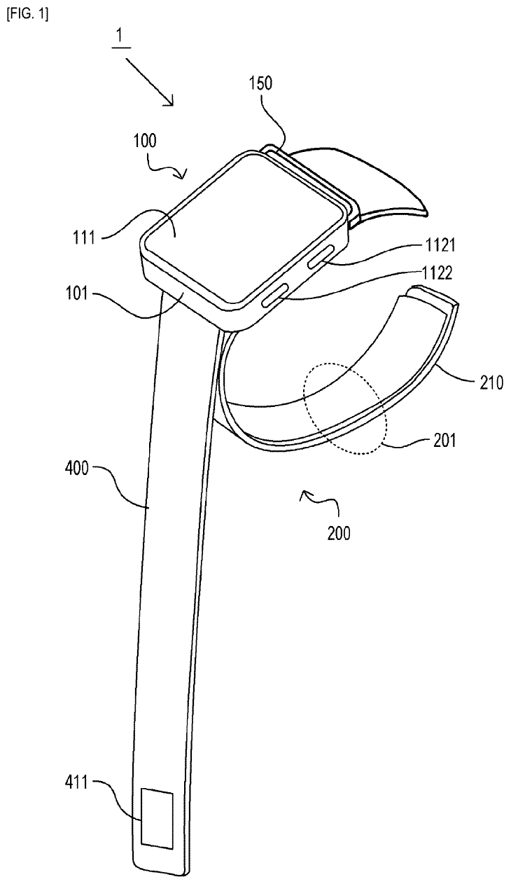

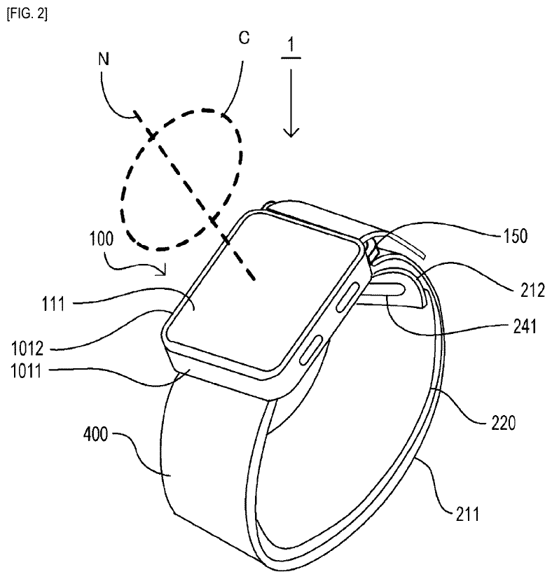

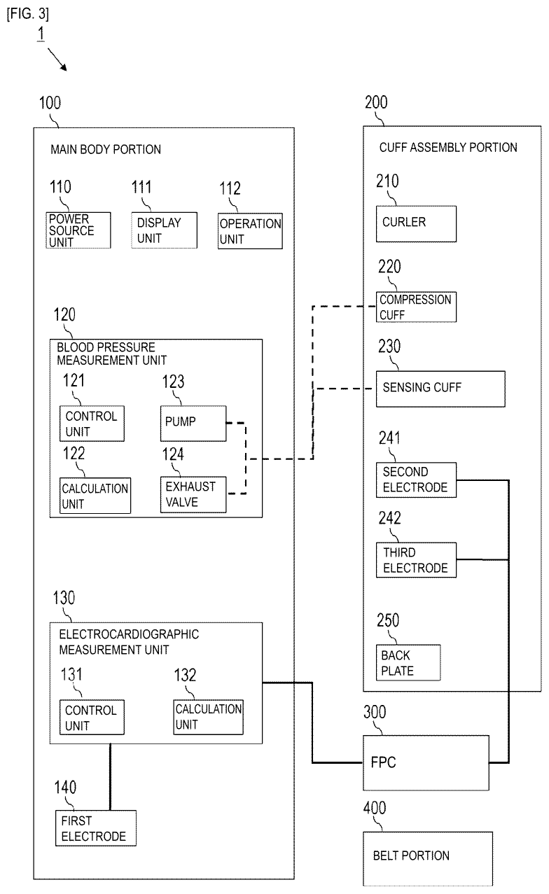

FIGS. 1 and 2 are schematic diagrams illustrating an external configuration of a biological information measurement device 1 according to the present example. FIG. 3 is a functional block diagram illustrating a functional configuration of the biological information measurement device 1 according to the present example.

As illustrated in FIGS. 1 to 3, the biological information measurement device 1 includes a main body portion 100, a cuff assembly portion 200, and a belt portion 400, and can measure a blood pressure value and an electrocardiographic waveform while being worn on a wrist T of a subject. The belt portion 400 includes a hook-and-loop fastener 411 having a hook. The main body portion 100 is provided with a belt loop portion 150 having an annular belt loop through which the belt portion 400 is inserted. When the biological information measurement device 1 is mounted, the belt portion 400 is wound around the wrist T and then inserted through the belt loop portion 150, and the hook-and-loop fastener 411 is attached to an arbitrary position of the belt portion 400 (where a loop with which a hook is engaged is formed), thereby fixing the biological information measurement device 1. The biological information measurement device 1 includes a flexible printed circuit (FPC) 300 (not illustrated in FIG. 1 and FIG. 2) on which wiring for electrically connecting an electrocardiographic measurement unit 130 of the main body portion 100 and a second electrode 241 and a third electrode 242 of the cuff assembly portion 200 to each other is disposed. Here, the wrist T corresponds to the measurement site of the present invention, and the second electrode 241 corresponds to the second electrode of the present invention. The main body portion 100 corresponds to the main body portion of the present invention, and the belt portion 400, the hook-and-loop fastener 411, and the belt loop portion 150 correspond to the fixing portion of the present invention.

As illustrated in FIG. 3, the main body portion 100 includes a casing 101, a power source unit 110, a display unit 111, an operation unit 112, a blood pressure measurement unit 120, an electrocardiographic measurement unit 130, and a first electrode 140. Here, as will be described below, the first electrode 140 includes the entire circumference of the casing 101 of the main body portion 100 and operation buttons 1121a and 1122a constituting the operation unit 112. Here, the first electrode 140 corresponds to the first electrode of the present invention, and the operation buttons 1121a and 1122a correspond to the instruction input portion of the present invention.

The power source unit 110 includes a battery that supplies the power required for operation of the device. The battery may be, for example, a secondary battery such as a lithium ion battery, or a primary battery.

The display unit 111 may include a display device such as a liquid crystal display, and may be provided with an LED indicator or the like. Specifically, the operation unit 112 includes the operation buttons 1121a and 1122a disposed on the side surface of the casing 101 of the main body portion 100 independently of the casing 101. The display unit 111 such as a touch panel display and the operation unit 112 may be integrated.

The blood pressure measurement unit 120 is a functional unit that controls the cuff assembly portion 200 described below, and measures the blood pressure of the user based on information obtained by the cuff assembly portion 200, and includes a control unit 121, a calculation unit 122, a pump 123, and an exhaust valve 124. The control unit 121 and the calculation unit 122 include, for example, a central processing unit (CPU) or the like, and although not illustrated, may include a storage unit including a random access memory (RAM) or the like.

The control unit 121 is a functional unit for controlling the blood pressure measurement unit 120, and controls the cuff pressure of the cuff assembly portion 200 via the calculation unit 122, the pump 123, and the like, and acquires information for measuring the user's blood pressure from the artery on the wrist T to which the biological information measurement device 1 is attached. The calculation unit 122 measures the blood pressure value based on the information obtained in this manner. The pump 123 and the exhaust valve 124 are mechanisms for supplying and exhausting air to and from a compression cuff 220 and a sensing cuff 230 described below. Here, the blood pressure measurement control unit of the present invention includes the control unit 121.

The electrocardiographic measurement unit 130 is a functional unit that measures an electrocardiographic waveform of the user based on a potential difference between the first electrode 140 and the second electrode 241 in contact with a surface of the human body, and includes a control unit 131 and a calculation unit 132. The control unit 131 and the calculation unit 132 include the above-described CPU or the like. From the viewpoint of hardware, the control unit 131 and the calculation unit 132 may have the same configuration as the control unit 121 and the calculation unit 122 of the blood pressure measurement unit 120. In this case, the electrocardiographic measurement control unit of the present invention includes the control unit 131.

Each of the blood pressure measurement unit 120 and the electrocardiographic measurement unit 130 includes an AD conversion circuit, an amplifier, a filter, and the like (not illustrated) in addition to the CPU, the RAM, and the like described above. However, these are configured by a known technique, and thus description thereof is omitted.

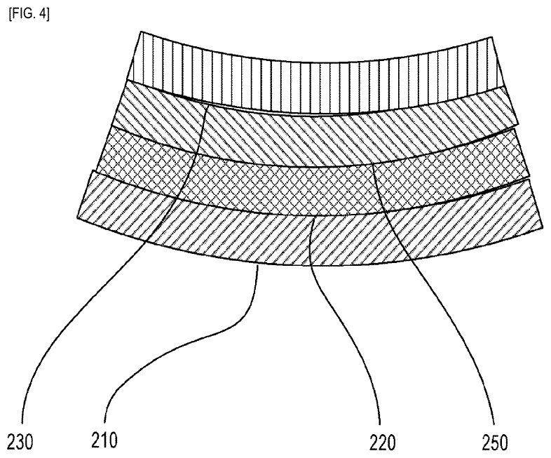

The cuff assembly portion 200 includes a curler 210, the compression cuff 220, the sensing cuff 230, the second electrode 241, the third electrode 242, and a back plate 250. The curler 210 is a base member for holding the compression cuff 220. FIG. 4 is a cross-sectional view schematically illustrating an internal structure of a region surrounded by a dotted line in FIG. 1 in the cuff assembly portion 200. The cuff assembly portion 200 has a configuration in which the compression cuff 220, the back plate 250, and the sensing cuff 230 are stacked in this order with the curler 210 as the outermost layer. The second electrode 241 and the third electrode 242 are connected to the FPC 30 with a conductive wire provided, and the PFC 300 is connected to the control unit 131 of the main body portion 100 (not illustrated) and thus functions as wiring for electrically connecting the control unit 131 and each electrode.

The compression cuff 220 is inflated by the air sent from the pump 123 to tighten the wrist T, thereby applying external pressure to an artery (not illustrated) existing in the wrist T. The sensing cuff 230 (not illustrated) is a fluid bag for detecting the pressure applied to the site compressed by the compression cuff 220, and measures the pressure applied to the site of compression by detecting the internal pressure with a pressure gauge (not illustrated) in a state where a small amount of air is in the sensing cuff 230. The back plate 250 (not illustrated) is a flexible flat plate member disposed between the compression cuff 220 and the sensing cuff 230, and suppresses excessive bending of the sensing cuff 220 when compressed by the compression cuff 230, thereby equalizing the pressure distribution in the sensing cuff 230.

As will be described below, the second electrode 241 and the third electrode 242 are disposed in the vicinity of a tip end portion 212a of a second curler portion 212, which is shorter in the extending direction of the curler 210. The arrangement of the second electrode 241 and the third electrode 242 is not limited to this, and they can be disposed at a position where they can be in contact with the body surface of the subject together with the first electrode 140 and an electrocardiographic waveform can be detected. The second electrode 241 functions as an electrode for measuring an electrocardiographic waveform, and the third electrode 242 functions as a GND (ground) electrode for setting a reference potential.

### Structure of Cuff Assembly Portion

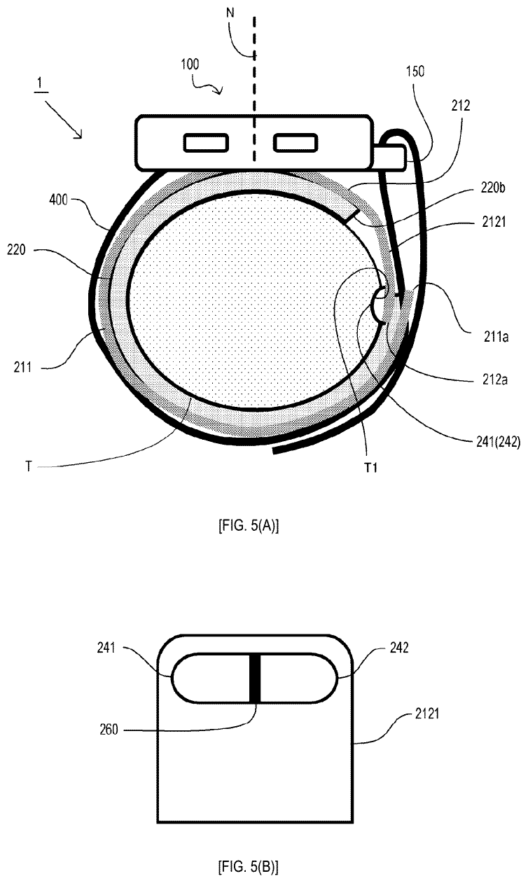

The structure of the cuff assembly portion 200 will be described with reference to FIG. 5(A) illustrating a state where the biological information measurement device 1 is attached to the wrist T of the user. In the present example, the compression cuff 220 is provided along the extending direction of the C-shaped curler 210 (i.e., the direction of rotation around the wrist T). The curler 210 includes a first curler portion 211 that is longer in the extending direction and the second curler portion 212 that is shorter in the extending direction with reference to the position where the main body portion 100 is provided. The first curler portion 211 extends from the main body portion 100 located on the back side of the wrist T so as to cover the artery side of the wrist T. On the other hand, the second curler portion 212 extends to the side opposite to the first curler portion 211 with respect to the circumferential direction of the wrist T. The compression cuff 220 is provided continuously from the vicinity of the end portion 211a of the first curler portion 211 of the curler 210 along the curler 210 and also along the second curler portion 212, but a tip end portion 220b of the compression cuff 220 in the circumferential direction (the extending direction of the second curler portion 212) is positioned apart from the tip end portion 212a of the curler 210 in the extending direction of the second curler portion 212. The second electrode 241 and the third electrode 242 are provided in the vicinity of the tip end portion 212a of an electrode supporting portion 2121 extending beyond the tip end portion 220b of the compression cuff 220 of the second curler portion 212. FIG. 5(B) is a diagram of the second electrode 241 and the third electrode 242 as viewed from the inside of the electrode supporting portion 2121 (the side in contact with the wrist T), and the second electrode 241 and the third electrode 242 are disposed side by side in a direction orthogonal to the circumferential direction. An insulating separator 260 is disposed between the second electrode 241 and the third electrode 242.

### Electrode Configuration of Main Body Portion

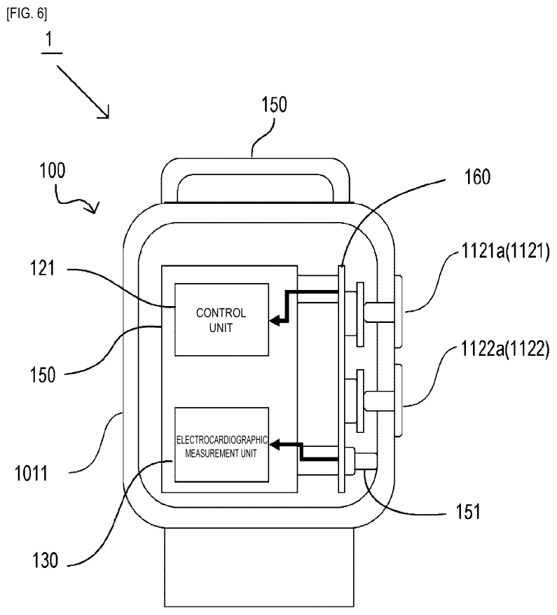

FIG. 6 is a schematic diagram for describing the electrical connection of the first electrode 140, a switch 1121, and a switch 1122 in the main body portion 100. A main circuit board 160 on which a CPU and the like constituting the blood pressure measurement unit 120, the electrocardiographic measurement unit 130, and the like of the main body portion 100 are mounted is accommodated in the casing 101. In the biological information measurement device 1, a side wall portion 1011 which surrounds the entire circumference of the main body portion 100 from the outer circumference side in a circumferential direction C (see FIG. 2) when a direction N (see FIGS. 2 and 5(A)) facing the wrist T of the main body portion 100 of the biological information measurement device 1 worn on the wrist T is defined as an axial direction in the casing 101 for housing the main body portion 100 is composed of a conductive member, and the entire circumference of the side wall portion 1011 of the casing 101 functions as the first electrode 140 by being electrically connected to the electrocardiographic measurement unit 130 via a conductive electrode connection portion 161. At this time, a frame portion 1012 surrounding the display unit 111 continuously with the side wall portion 1011 may be similarly constituted by a conductive member, and the first electrode 140 may be constituted by including the side wall portion 1011 and the frame portion 1012. The operation button 1121a of the switch 1121 and an operation button 1122a of the switch 1122 constituting the operation unit 112 are disposed on the side wall portion 1011 of the casing 101. The switches 1121 and 1122 are brought into contact with and separated from a switch board 170 through an insulating material by a user's operation of the operation buttons 1121a and 1122a. The switch board 170 is electrically connected to the control unit 121 and the like provided on the main circuit board 160, and a signal generated by switching of the switches 1121 and 1122 is input thereto. The operation buttons 1121a and 1122a are formed of a conductive member and are electrically connected to the side wall portion 1011, and the first electrode 140 includes the side wall portion 1011 and the operation buttons 1121a and 1122a. In the biological information measurement device 1 illustrated in FIGS. 1 and 2, the main body portion 100 has a substantially rectangular parallelepiped shape, and the side wall portion 1011 surrounding the entire circumference of the main body portion 100 from the outer circumferential side in the circumferential direction C when the direction N facing the wrist T of the main body portion 100 is defined as the axial direction has a rectangular shape, but may have a cylindrical shape that is short in the axial direction N of the main body portion 100, and in this case, the side wall portion 1011 has a circular shape. The shape of the main body portion 100 is not limited to these shapes, and the side wall portion 1011 can be formed in an appropriate shape in accordance with the shape of the main body portion 100.

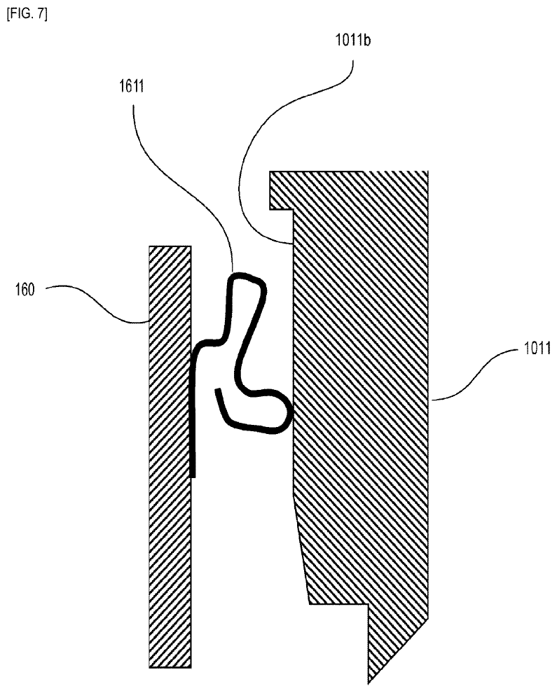

FIG. 7 is a diagram illustrating a specific configuration of the side wall portion 1011 of the casing 101, the main circuit board 160, and the electrode connection portion 161. Here, a conductive leaf spring 1611 is disposed between an inner surface 1011b of the side wall portion 1011 of the casing 101 and the main circuit board 160. The elastically deformed leaf spring 1611 is brought into pressure contact with the inner surface 1011b of the side wall portion 1011 and the main circuit board 160 by a restoring force, thereby establishing an electrical connection between the side wall portion 1011 of the casing 101 and the main circuit board 160.

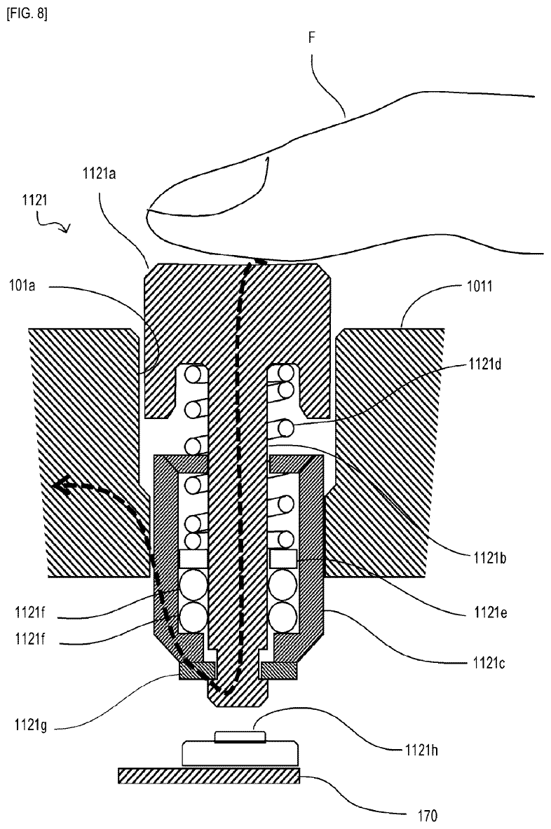

FIG. 8 is a diagram specifically illustrating the electrical connection between the switch 1121 (or 1122) and the casing 101. Although the switch 1121 will be mainly described below, the switch 1122 is also configured in the same manner.

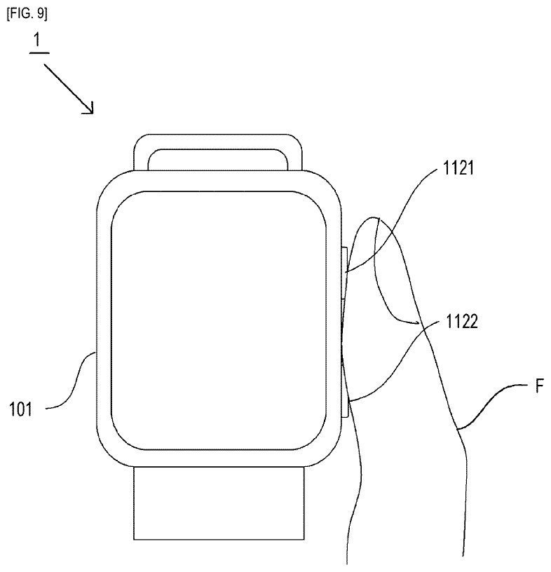

The switch 1121 includes the operation button 1121a functioning as a key top, a rod-shaped plunger 1121b extending from the operation button 1121a, a housing 1121c supporting the plunger 1121b, a spring 1121d, a washer 1121e, an O-ring 1121f, a washer 1121g, a tact switch 1121h, and the switch board 170. Here, the washer 1121g is fitted into a groove provided on the outer circumference surface of the distal end of the plunger 1121b. Two O-rings 1121f, which are attached to the outer periphery of the plunger 1121b, are disposed inside the housing 1121c on the distal end side. Further, the washer 1121e for supporting the distal end side of the spring 1121d wound around the outer periphery of the plunger 1121b is disposed on the proximal end side of the O-ring 1121f. The proximal end of the spring 1121d is supported by an operation button 1121a. The housing 1121c is fixed to a switch hole portion 101a of the casing 101. When the user presses the operation button 1121a against the elastic force of the spring 1121d, the plunger 1121b presses the tact switch 1121h. When the user releases the push of the operation button 1121a, the operation button 1121a is pushed back by the elastic return of the spring 1121d, and the plunger 1121b is separated from the tact switch 1121h. Thus, the operation button 1121a and the plunger 1121b reciprocate with respect to the housing 1121c. In the biological information measurement device 1, the operation button 1121a, the plunger 1121b, the washer 1121g, and the housing 1121c are formed of conductive members. Thus, the user's finger F touching the operation button 1121a is electrically connected to the side wall portion 1011 of the casing 101 by the plunger 1121b, the washer 1121g, and the housing 1121c. In FIG. 8, an electrically conductive path from the user's finger F to the side wall portion 1011 of the casing 101 is indicated by a broken line. In this manner, since not only the side wall portion 1011 of the casing 101 but also the operation buttons 1121a and 1122a function as the first electrode 140, as illustrated in FIG. 9, even when the user touches the switch 1121 or 1122 together with the casing 101, the contact surface between the finger F of the user and the first electrode 140 can be stabilized.

### Measurement of Biological Information

To measure biological information by the biological information measurement device 1 having the above-described configuration, first, the cuff assembly portion 200 and the belt portion 400 are wound around the wrist T such that the main body portion 100 faces the back of the hand. Then, the belt portion 400 is passed through the belt loop portion 150 and folded back, and the hook-and-loop fastener 411 of the belt portion 400 is attached to an arbitrary position of the belt portion 400, and the biological information measurement device 1 is mounted and fixed on the wrist T. At this time, the sensing cuff 230 is mounted so as to be located on the palm side of the wrist T.

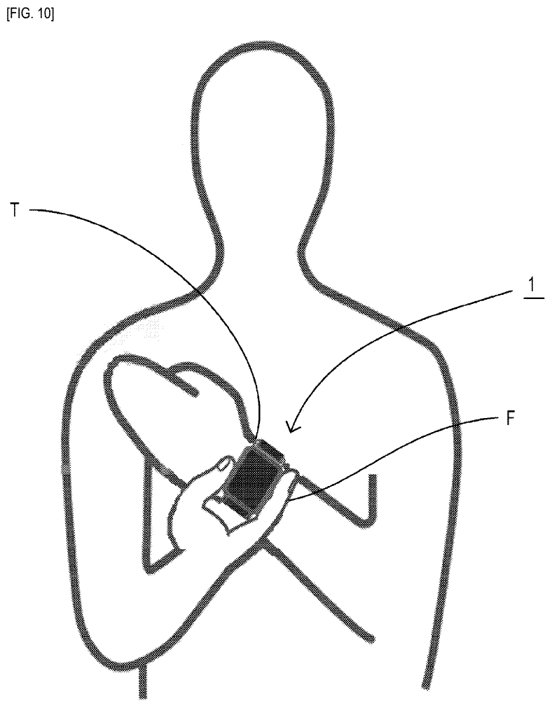

Then, the biological information measurement device 1 is held at the height of the heart, and the finger F of the hand (the right hand in FIG. 10) opposite to the hand on which the biological information measurement device 1 is mounted touches the casing 101 and operates the operation button 1121a (or 1122a), thereby instructing the start of the measurement of the blood pressure. Specifically, air is injected into the compression cuff 220 and expanded to compress (the artery of) the wrist T, the artery is blocked to temporarily stop the blood flow, and then the air is gradually discharged from the compression cuff 220 and contracted to release the compression and return the blood flow of the artery, and the pressure at that time is measured by the sensing cuff 230. That is, the blood pressure measurement is performed by a so-called oscillometric method.

When the wrist T is compressed by the compression cuff 220 during the blood pressure measurement, the second electrode 241 and the third electrode 242 are in contact with (pressed against) surfaces T1 and T2 (see FIG. 5(A)) of the wrist T. Therefore, the electrocardiographic waveform can be measured by the so-called I induction method based on the potential difference between the first electrode 140 and the second electrode 241 provided on the casing 101 of the main body portion 100 with the finger not wearing the biological information measurement device 1. Here, the finger F which is not wearing the biological information measurement device 1 corresponds to the first site of the present invention, and the surface T1 of the wrist T which is different from the finger F corresponds to the second portion of the present invention.

When the subject takes the measurement posture as illustrated in FIG. 10, the casing 101 surrounding the main body portion 100 of the biological information measurement device 1 is formed as the first electrode 140 over the entire circumference, and the first electrode 140 is formed including the operation buttons 1121a and 1122a electrically connected to the casing 101, so that even when the subject operates the operation button 1121a (or 1122a) while touching a part of the casing 101, a change in the contact state with the first electrode 140 is suppressed, a stable contact state can be realized, and the electrocardiographic waveform can be measured with high accuracy. In addition, since the degree of freedom of the position where the subject touches the first electrode 140 is high, unnecessary force is not applied in taking the measurement posture, and noise can be reduced.

As described above, according to the biological information measurement device 1 of the present example, the blood pressure value and the electrocardiographic waveform can be simultaneously and accurately measured by the portable device of a type to be attached to the wrist T.

### Modified Example

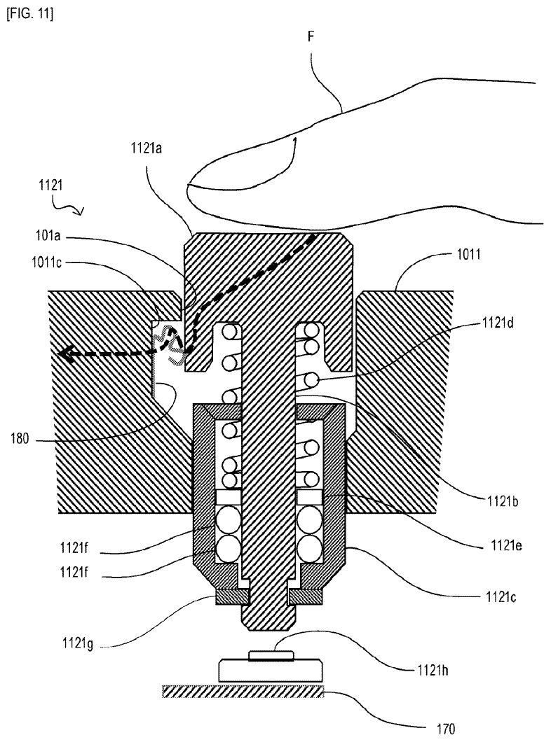

FIG. 11 is a diagram illustrating a modified example of the electrical connection between the switch 1121 (or 1122) and the casing 101.

The configuration of the switch 1121 is the same as that of Example 1, and includes the operation button 1121a, the rod-shaped plunger 1121b extending from the operation button 1121a, the housing 1121c supporting the plunger 1121b, the spring 1121d, the washer 1121e, the O-ring 1121f, the washer 1121g, the tact switch 1121h, and the switch board 170. Here, a groove portion 1011c is provided at a position of a switch hole portion 101a of the side wall portion 1011 of the casing 101 facing the side surface of the operation button 1121a, and an electrode connection portion plate spring 180 made of a conductive member is disposed. The elastically deformed electrode connection portion plate spring 180 is brought into pressure contact with the casing 101 and the operation button 1121a by the restoring force, thereby establishing electrical connection between the operation button 1121a and the side wall portion 1011 of the casing 101. As a result, the finger F of the user touching the operation button 1121a and the side wall portion 1011 of the casing 101 are electrically connected by the electrode connection portion plate spring 180. In FIG. 11, an electrically conductive path from the user's finger F to the casing 101 is indicated by a broken line.

### Example 2

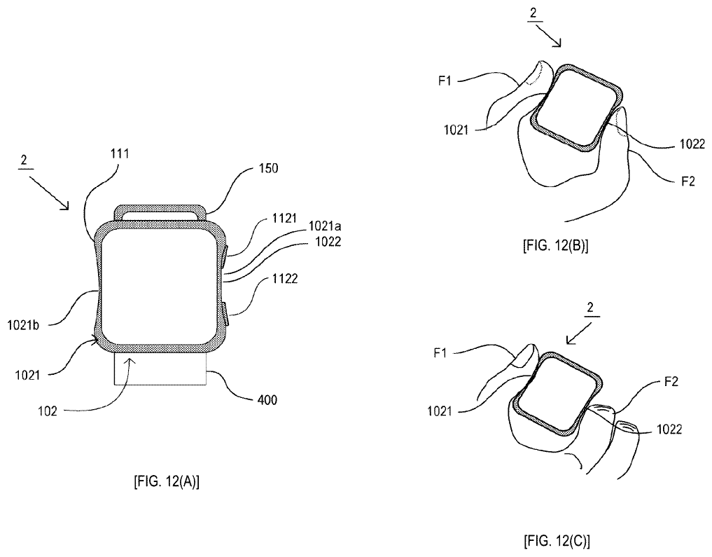

FIGS. 12(A) to 12(C) are diagrams of a biological information measurement device 2 according to Example 2 as viewed from the front of the display unit 111. The same components as those of the biological information measurement device 1 according to Example 1 are denoted by the same reference numerals, and detailed description thereof will be omitted.

The biological information measurement device 2 according to Example 2 has the same configuration as that of the biological information measurement device 1 according to Example 1except for the shape of a casing 102. Also in the present example, the first electrode 140 is configured to include at least a side wall portion 1021 that surrounds the entire circumferential direction of the main body portion 100 of the biological information measurement device 1 worn at least on the wrist T from the outer circumference side in the circumferential direction C (see FIG. 2) when the direction N (see FIGS. 2 and 5(A)) facing the wrist T is defined as the axial direction of the main body portion 100. In the casing 102 of the biological information measurement device 2, as illustrated in FIG. 12(A), a side wall portion 1021a on which the operation buttons 1121 and 1122 are provided and a side wall portion 1021b opposed to the side wall portion 1021a are formed in a curved surface that is convex inward at the center in the longitudinal direction, and both side wall portions 1021a and 1021b of the casing 102 are formed in a constricted shape. That is, the side wall portion 1021 of the biological information measurement device 2 includes side wall portions 1021a and 1021b which are concave toward the inner circumference side in the circumferential direction C (see FIG. 2) when the direction N facing the wrist T of the main body portion 100 of the biological information measurement device 1 worn on the wrist T is defined as the axial direction. The curved shape of the side wall portions 1021a and 1021b is not limited to the shape illustrated in FIG. 12(A). Here, the side wall portions 1021a and 1021b correspond to the concave side wall portions of the present invention.

By forming the casing 102 of the biological information measurement device 2 in such a shape that the side wall portions 1021 and 1022 are constricted, when the user touches the side wall portions 1021 and 1022 of the casing 102 with a thumb F1 and an index finger F2 of the right hand, the thumb F1 and the index finger F2 are less likely to shift, and the contact surfaces of the finger, the casing 102, and the switches 1121 and 1122 are stabilized.

Making the curvature (concave shape) of the side wall portions 1021 and 1022 gradual causes, when the user touches the side wall portions 1021 and 1022 with his/her finger, the position where the user touches the side wall portions 1021 and 1022 with his/her finger not to be limited. When the user touches the side wall portions 1021 and 1022 of the casing 102 with the thumb F1 and the index finger F2 of the right hand, the user can touch with the belly of the thumb F1 and the belly of the index finger F2 as illustrated in FIG. 12(B), or with the belly of the thumb F1 and the second joint portion of the index finger F2 as illustrated in FIG. 12(C). Making the curvature of the side wall portions 1021 and 1022 gradual in this manner allows the user to touch the casing 102 in various ways, thus the electrocardiogram can be measured in a posture in which it is difficult to apply a force without being forced into an unreasonable posture in which a force is applied by limiting the position where the user touches the casing 102.

### Modified Example

In Example 2, the side wall portion 1021 of the casing 102 is provided with the side wall portions 1021a and 1021b having a constricted shape, and the center portion in the circumferential direction surrounding the main body portion 100 of the side wall portions 1021a and 1021b respectively constituting one side surface of the substantially rectangular parallelepiped casing 102 is formed into a curved shape that is convex toward the inner circumference side, but a fine uneven portion may be formed on the entire surface of the side wall portion 1021 or a part thereof. Since the uneven portion increases the frictional resistance between the finger F and the user's finger F, the finger F is less likely to slip relative to the first electrode 140 including the side wall portion 1021, and the contact state between the finger F and the first electrode 140 is stabilized. Similarly, the fine uneven portions may be formed on the surfaces of the operation buttons 1121a and 1122a.

The uneven portion may be formed by alternately arranging concave portions and convex portions in the circumferential direction C as in the bezel of a wristwatch, or by roughening the surface of the side wall portion 1021, and the configuration of the uneven portion can be appropriately selected.

### Example 3

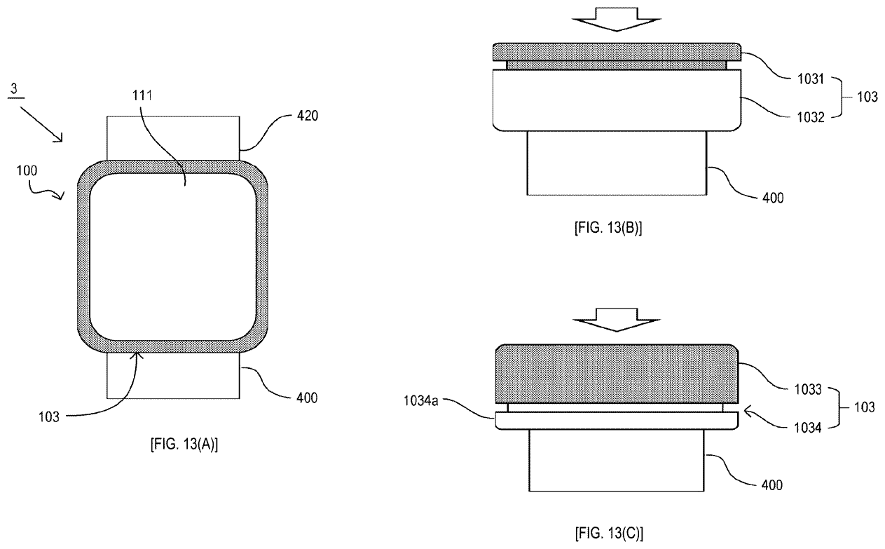

FIG. 13(A) is a diagram of a biological information measurement device 3 according to Example 3 as viewed from the front of the display unit 111. The same components as those of the biological information measurement device 1 according to Example 1 are denoted by the same reference numerals, and detailed description thereof will be omitted.

The biological information measurement device 3 according to Example 3 has the same configuration as that of the biological information measurement device 1 according to Example 1 except for the configuration of the first electrode 140 and the operation button. Also in the biological information measurement device 3 according to Example 3, the first electrode 140 is configured to include at least a side wall portion 1031 that surrounds the entire circumferential direction of the main body portion 100 of the biological information measurement device 3 worn at least on the wrist T from the outer circumference side in the circumferential direction C (see FIG. 2) when the direction N (see FIGS. 2 and 5(A)) facing the wrist T is defined as the axial direction of the main body portion 100. The side wall portion 1031 included in a casing 103 functions as the first electrode 140 and also functions as an operation button.

In the biological information measurement device 3, the side wall portion 1031 constituting a bezel portion surrounding the display unit 111 is formed. The side wall portion 1031 is formed of a conductive member and functions as the first electrode 140. The side wall portion 1031 is attached to a casing main body 1032 as a movable portion so as to also function as an operation button. For example, in the biological information measurement device 3 illustrated in FIG. 13(B), the side wall portion 1031 surrounding the display unit 111 is supported so as to reciprocate with respect to the rectangular parallelepiped casing main body 1032 to which the belt portion 400 is attached. The side wall portion 1031 functions as an operation button by pushing the side wall portion 1031 in the arrow direction with respect to the casing main body 1032. In the biological information measurement device 3 illustrated in FIG. 13(C), a side wall portion 1033, which is bent from the frame portion 1012 of the display unit 111 and formed so as to cover the side surface of the casing main body 1034, is supported so as to reciprocate by the casing main body 1034 from which the flange-shaped cover portion 1034a is exposed. Also in this case, the side wall portion 1033 is made of a conductive member and functions as the first electrode 140. The side wall portion 1033 functions as an operation button by pushing the side wall portion 1033 in the arrow direction with respect to the casing main body 1034. That is, the side wall portions 1031 and 1033 are the side wall portions of the present invention, and correspond to the integrally provided instruction input portion.

In this manner, since the side wall portions 1031 and 1033 also function as operation buttons, there is no need for the user to touch a specific part for instruction input, and there is a degree of freedom in the position where the user touches the side wall portions 1031 and 1033 for electrocardiographic measurement.

The side wall portions 1031 and 1033 of the biological information measurement device 3 according to Example 3 may be formed into the concave shape according to Example 2.

### REFERENCE NUMERALS LIST

1 Biological information measurement device
100 Main body portion
101 Casing
121, 131 Control unit
1011 Side wall portion
140 First electrode
241 Second electrode
400 Belt portion
1121a, 1122a Operation button

What is claimed is:

1. A biological information measurement device for measuring a blood pressure and an electrocardiographic waveform of a subject, the biological information measurement device comprising:
a blood pressure measurement control unit configured to control measurement of a blood pressure of a measurement site of the subject;
a first electrode to be in contact with a first site of the subject;
a second electrode to be in contact with a second portion of the subject different from the first site;
an electrocardiographic measurement control unit configured to control measurement of an electrocardiographic waveform of the subject through the first electrode and the second electrode;
a main body portion including the blood pressure measurement control unit and the electrocardiographic measurement control unit;
an instruction input portion operated by the subject to input an instruction; and
a fixing portion configured to fix the main body portion to the measurement site, wherein
the main body portion includes a casing including a side wall portion surrounding an entire circumference of the main body portion from an outer circumference side when a direction facing the measurement site is defined as an axial direction, and
the first electrode includes a surface of the side wall portion along the direction facing the measurement site and the instruction input portion.

2. The biological information measurement device according to claim 1, wherein the instruction input portion is provided independently of the side wall portion.

3. The biological information measurement device according to claim 1, wherein the instruction input portion includes the side wall portion.

4. The biological information measurement device according to claim 1, wherein the side wall portion includes a concave side wall portion which is concave toward an inner circumference side when the direction facing the measurement site is defined as the axial direction.

5. The biological information measurement device according to claim 1, wherein an uneven portion is formed on a surface of the side wall portion.

6. The biological information measurement device according to claim 1, wherein the instruction is an instruction for the measurement of the blood pressure.

7. A biological information measurement device for measuring a blood pressure and an electrocardiographic waveform of a subject, the biological information measurement device comprising:
a blood pressure measurement control unit configured to control measurement of a blood pressure of a measurement site of the subject;
a first electrode to be in contact with a first site of the subject;
a second electrode to be in contact with a second portion of the subject different from the first site;
an electrocardiographic measurement control unit configured to control measurement of an electrocardiographic waveform of the subject through the first electrode and the second electrode;
a main body portion including the blood pressure measurement control unit and the electrocardiographic measurement control unit;
an instruction input portion operated by the subject to input an instruction; and
a fixing portion configured to fix the main body portion to the measurement site, wherein
the main body portion includes a casing including a side wall portion surrounding an entire circumference of the main body portion from an outer circumference side when a direction facing the measurement site is defined as an axial direction,
the first electrode includes the side wall portion and the instruction input portion, and
the instruction input portion includes the side wall portion.

8. The biological information measurement device according to claim 7, wherein the instruction is an instruction for the measurement of the blood pressure.
